(19) United States
(12) Patent Application Publication
(10) Pub. No.: US 2024/0382728 A1
(43) Pub. Date: Nov. 21, 2024

(54) LAYERED MATERIAL FOR MUCOADHESION, AND PATCH AND MICRONEEDLE PATCH COMPRISING THE SAME

(71) Applicant: Win Coat Corporation, Hsinchu City (TW)

(72) Inventors: My-Huyen NGUYEN, Hsinchu City (TW); Hsiu-Feng YEH, Hsinchu City (TW); Hsiao-Chun CHOU, Hsinchu City (TW); Jie-Wei HSU, Hsinchu City (TW); Yi-Jyun LIAO, Hsinchu City (TW); Hsin-Kuo CHANG, Hsinchu City (TW); Ta-Jo LIU, Hsinchu City (TW)

(21) Appl. No.: 18/244,521

(22) Filed: Sep. 11, 2023

(30) Foreign Application Priority Data
May 16, 2023 (TW) 112118180

(51) Int. Cl. A61M 37/00

(52) U.S. Cl. CPC A61M 37/0015; A61M 2037/0023; A61M 2037/0053; A61M 2210/0631

(57) ABSTRACT

The present invention relates to a layered material for mucoadhesion, comprising a modified acrylic polymer and hydroxypropyl methylcellulose, wherein the weight ratio of the modified acrylic polymer: hydroxypropyl methylcellulose is 1:4 to 4:1, and the layered material has a thickness of 0.0001 millimeters to 100 millimeters. The layered material of the present invention has mucoadhesion ability, so it can be prepared into a patch or a needle patch and help the fixation of the patch or the needle patch, and this is advantageous for the controlled-release of drugs. The present invention also relates to a patch comprising the layered material. In addition, the present invention relates to a microneedle patch comprising a needle layer, a base layer and a backing layer, wherein the backing layer comprises the aforementioned layered material and benefits the fixation of the microneedle patch. This microneedle patch is suitable for lesions on mucosa.

### CROSS-REFERENCE TO RELATED APPLICATION

Pursuant to 35 U.S.C. § 119(a), this application claims the benefits of the priority to Taiwan Patent Application No. 112118180, filed May 16, 2023. The contents of the prior application are incorporated herein by entirety.

### BACKGROUND OF THE INVENTION

### 1. Field of the Invention

The present invention relates to a layered material for mucoadhesion and a patch comprising the aforementioned layered material, and particularly, relates to a microneedle patch for mucosa.

### 2. Description of the Prior Arts

Gums and alveolar bone naturally lose with age, leading to gingival recession. Gingival recession may be early-onset gingival recession due to congenital fragile gums, or gingival recession resulting from diseases or unhealthy living habits, such as periodontal disease, bruxism, denture compression, improper brushing habits, and smoking. People may improve gingival recession through early treatment of periodontal disease, replacement of ill-fitting denture, adjustment of improper or excessive brushing habits, or quitting smoking; or they may seek an orthodontist's assistance for teeth alignment, and the correction of teeth arrangement makes the teeth and tooth roots easier to clean. The treatment of periodontal disease includes surgical and mechanical root surface debridement for teeth, and the treatment also includes systemic or tropical administration of antibiotics for infection treatment. Also, mucoadhesive tablets, toothpaste, and mouthwash can be used to deliver drugs, yet these approaches are found difficult to control the dosage that enters the lesion.

Some diseases, such as erythema multiforme and systemic lupus erythematosus, would attack body parts such as eyes, ears, and noses, causing mucosa damage. Mucosa-related symptoms mainly include erosion, ulceration, and/or white lesion. Erosion is a lesion of exfoliation of the mucosal epithelium, and ulceration is a lesion extending from the mucosal epidermis to the lamina propria below. Erosive and ulcerative diseases may be a common disease such as aphthous stomatitis, or a rare disease such as pemphigus. Furthermore, these diseases may be idiopathic, immune, infectious, and malignant, which may cause symptoms such as diffuse gastric mucosal petechiae and erosion, and may furtherly induce cold sores, oral cancer, angular cheilitis, dental stomatitis, and the like. Recent studies have discovered that drug delivery via mucosa is especially suitable for systemic drug administration. There are many merits of drug delivery via mucosa. It is a non-invasive treatment, which is convenient and works quickly; and it avoids hepatic first-pass metabolism, saving drugs from the degradation by enzymes before entering the liver.

The advantages of the mucosal drug delivery system (MDDS) include its safety, easy-to-use, capability of self-administration at home, and painlessness. Meanwhile, the microneedle array patches over the parenteral route are advantageous since the microneedle array patches are small, and simpler to use than the syringe, and they can reduce pain and fear for patients, and can deliver an extremely small amount of drug into the body without causing scarring reactions. Compared to the conventional transdermal patches, the drugs delivered by microneedle array patches can be delivered more effectively into the body by concentrating the drugs on microneedles which penetrate the mucosa through skin directly. However, the mucosa would secrete water-containing mucus, making it difficult for adhesion and consequently difficult to fix the drugs on the lesion. Therefore, it is required to develop a material which can adhere to the mucosa with good fixation.

### SUMMARY OF THE INVENTION

In view of this, an objective of the present invention is to provide a layered material for mucoadhesion comprising a modified acrylic polymer and hydroxypropyl methylcellulose (HPMC), wherein a weight ratio of the modified acrylic polymer: hydroxypropyl methylcellulose (HPMC) is 1:4 to 4:1, and the layered material has a thickness of 0.0001 millimeters (mm) to 100 mm.

Another objective of the present invention is to provide a patch comprising a supportive layer and an adhesive layer, wherein the adhesive layer comprises the aforementioned layered material and the adhesive layer is disposed on one side of the supportive layer.

Still another objective of the present invention is a microneedle patch comprising a needle layer, a base layer, and a backing layer, wherein the backing layer comprises the aforementioned layered material, the base layer is disposed between the needle layer and the backing layer, and the backing layer comprises at least one protrusion part which is free of connection with the base layer.

The term “bioadhesion” is defined as the state in which at least one biomaterial in nature and another material are held together for a period of time by interfacial forces. “Bioadhesion” can be divided into three types: (1) an adhesion between two types of biomaterials, e.g., platelet aggregation or wound healing, (2) an adhesion of a biomaterial to an artificial substrate, e.g., cell adhesion to a cell-culture dish and biofilm formation on a prosthetic device or an implant, and (3) an adhesion of an artificial material to a biosubstrate, e.g., adhesion of an artificial gel to a soft tissue and adhesion of a sealant to tooth enamel. Adhering a drug carrying system to a specific position of an organism (e.g., a biological surface) is a type of bioadhesion as well, wherein the biological surface may be a surface of an epidermal tissue or a mucosal tissue. Specifically, adhering the drug carrying system to the surface of the mucosal tissue is specifically called mucoadhesion. The aforementioned artificial substrate and the artificial material may also be made from natural materials.

The present invention has developed a layered material for mucoadhesion and may be applied to lesions located on the mucosa, in which the mucosa may be the mucosa of eyes, ears, respiratory tract (e.g., nose, trachea), digestive tract (e.g., mouth, lips, tongue, gum, stomach, anus), genital system (e.g., uterus), and the like.

The layered material for mucoadhesion of the present invention may be applied to the microneedle patch for mucosa, making the backing layer of the microneedle patch exhibit good mucoadhesion. The needle layer may penetrate the mucosa and deliver an effective component to the mucosa (e.g., functional components such as drugs, growth factors, nutrients). The backing layer capable for mucoadhesion comprises at least one protrusion part, and the at least one protrusion part may provide extra adhesion to the mucosa and help the fixation of the microneedle patch. The microneedle patch may continuingly deliver the drugs to the mucosa for a longer period of time without falling off easily.

In one of the embodiments, the aforementioned effective component may be a functional component used for preventive health care, repair promotion, moisture maintenance, anti-inflammation, and soothing, but is not limited thereto. Specifically, the effective component used for health care prevention and repair promotion may be selected as stem cell extracts and their related substances, placenta extracts, growth factors, proteins, vitamin B, zinc, iron, potassium, vitamin A, vitamin C, vitamin D, and vitamin E, but is not limited thereto. The moisturizing component may be selected as ceramide, lecithin, glycerol, polysaccharides, hyaluronic acid, sodium hyaluronate, proteins, collagen, elastin, peptides, amino acids, citrate, uric acid, urea, glucose, sucrose, fructose, glycogen, glucosamine, mucopolysaccharides, lactates, phosphates, and eythyl-di-2-pyrrolidone-5-carboxylate, but is not limited thereto. The anti-inflammatory or soothing component may be selected as grape extracts, green tea extracts, ginkgo extracts, soy extracts, pomegranate extracts, ginger extracts, yeast extracts, coix extracts, R-alpha-lipoic acid, glucan, coenzyme Q10, superoxide dismutase (SOD), vitamin C and its derivatives, vitamin E and its derivatives, anti-inflammation agents, steroidal anti-inflammatory materials, non-steroidal anti-inflammatory materials, anti-pain and anti-inflammation materials and their analogs, vitamin B and its derivatives, licorice extracts, triterpenoids, madecassoside, asiaticoside, mucosa coating agents, glutathione, and lycopenemia, but is not limited thereto.

In one of the embodiments, the aforementioned layered material is obtained by drying a composition liquid of a layered material comprising a modified acrylic polymer and hydroxypropyl methylcellulose, wherein a solid content of the composition liquid of the layered material is 0.5 weight percent (wt %) to 30 wt %, 0.5 wt % to 15 wt %, 0.5 wt % to 10 wt %, 1 wt % to 5 wt %, 1.5 wt % to 4.5 wt %, 2 wt % to 4 wt %, 2 wt % to 3.5 wt %, or 2 wt % to 3 wt %. By controlling the solid content of the layered material composition liquid, the present invention may substantially prevent the layered material from having an inferior hollow structure and being overly soft, which would cause deformation due to an unbearable external force.

According to the present invention, the aforementioned modified acrylic polymer may change the rheology and emulsion stability of the layered material. The modified acrylic polymer may also enable the formulation to be instantly moisturized, to swell quickly without stirring, and to offer adhesive capability to the layered material of the present invention.

In one of the embodiments, the weight ratio of the aforementioned modified acrylic polymer: hydroxypropyl methylcellulose is 1:3.5 to 3.5:1, 1:3 to 3:1, 1:2.5 to 2.5:1, or 1:2 to 2:1, or 1:2 to 1:1, or 1:1.5 to 1.5:1.

In one of the embodiments, the thickness of the aforementioned layered material is 0.01 mm to 10 mm, 0.05 mm to 5 mm, or 0.1 mm to 1 mm. In one of the embodiments, the thickness of the layered material is 0.01 mm to 0.05 mm, 0.01 mm to 0.004 mm, or 0.002 mm to 0.003 mm.

In one of the embodiments, the aforementioned modified acrylic polymer is a hydrophobically-modified cross-linked acrylate copolymer. In one of the embodiments, the aforementioned hydrophobically-modified cross-linked acrylate copolymer is an acrylates/C10-30 alkyl acrylate crosspolymer. In one of the embodiments, the aforementioned hydrophobically-modified cross-linked acrylate copolymer is a cross-linked acrylate copolymer modified by an allyl sucrose or an allyl pentaerythritol. In one of the embodiments, the aforementioned cross-linked acrylate copolymer modified by the allyl sucrose or the allyl pentaerythritol is carbopol.

In one of the embodiments, the aforementioned modified acrylic polymer has a viscosity of 30000 centipoise (cP) to 80000 cP, 40000 cP to 70000 cP, or 47000 cP to 67000 cP. According to the present invention, the viscosity of the aforementioned modified acrylic polymer refers to a property measured when the modified acrylic polymer is dissolved in water at 25° C. to formulate a 1% solution, and the measurement condition is abbreviated in the present specification as @25° C., 1% solution.

In one of the embodiments, the aforementioned hydroxypropyl methylcellulose may have a viscosity of 1 cP to 10000 cP. According to the present invention, the viscosity of the aforementioned hydroxypropyl methylcellulose refers to a property measured when the hydroxypropyl methylcellulose is dissolved in water at 20° C. to formulate a 2% solution, and the measurement condition is abbreviated in the present specification as @20° C., 2% solution.

In one of the embodiments, the aforementioned hydroxypropyl methylcellulose may be a high-viscosity hydroxypropyl methylcellulose, and its viscosity @20° C., 2% solution may be 400 cP to 10000 cP, 1000 cP to 8000 cP, 1500 cP to 6000 cP, 2000 cP to 5000 cP, 3000 cP to 4500 cP, or 3500 cP to 4000 cP. In one of the embodiments, the aforementioned hydroxypropyl methylcellulose is the high-viscosity hydroxypropyl methylcellulose, and the weight ratio of the modified arylic polymer: the hydroxypropyl methylcellulose may be 1:1.5 to 1:4, 1:2 to 1:3.5, 1:2.5 to 1:3, 2:1 to 3.5:1, or 2.5:1 to 3:1.

In one of the embodiments, the aforementioned hydroxypropyl methylcellulose may be a low-viscosity hydroxypropyl methylcellulose, and its viscosity @20° C., 2% solution may be 1 cP to 100 cP, 2 cP to 50 cP, 2 cP to 30 cP, 3 cP to 20 cP, 4 cP to 15 cP, or 4.5 cP to 6 cP. In one of the embodiments, the aforementioned hydroxypropyl methylcellulose is the low-viscosity hydroxypropyl methylcellulose, and the weight ratio of the modified arylic polymer: the hydroxypropyl methylcellulose may be 1.5:1 to 4:1, 2:1 to 3.5:1, or 2.5:1 to 3:1.

According to the present invention, both the aforementioned modified acrylic polymer and the hydroxypropyl methylcellulose are dissolvable or swellable materials. More specifically, the aforementioned modified acrylic polymer and the hydroxypropyl methylcellulose are biocompatible materials or biodegradable materials.

In one of the embodiments, the material of the aforementioned needle layer is a copper peptide and methyl vinyl ether-maleic anhydride copolymer. Specifically, the aforementioned needle layer is made of a needle composition liquid, and the needle composition liquid is a 20 wt % solution comprising the copper peptide and methyl vinyl ether-maleic anhydride copolymer, i.e., the needle composition liquid consists of 80 wt % of water and 20 wt % of a mixture of the copper peptide and methyl vinyl ether-maleic anhydride copolymer. The needle composition liquid has a viscosity of 40 cP measured at 25° C., 1 S⁻¹ and a surface tension of 30 dyne/cm.

In one of the embodiments, the material of the aforementioned base layer may comprise the aforementioned high-viscosity hydroxypropyl methylcellulose, the low-viscosity hydroxypropyl methylcellulose, and a polyvinylpyrrolidone-vinyl acetate copolymer, wherein the weight ratio of high-viscosity hydroxypropyl methylcellulose: low-viscosity hydroxypropyl methylcellulose is 1:0.1 to 1:1.2, and the amount of the polyvinylpyrrolidone-vinyl acetate copolymer is 0.25 wt % to 2 wt %.

In one of the embodiments, the weight ratio of the high-viscosity hydroxypropyl methylcellulose: low-viscosity hydroxypropyl methylcellulose in the aforementioned base layer is 1:0.2 to 1:1, preferably 1:0.2 to 1:0.8, more preferably 1:0.3 to 1:0.7.

The amount of the polyvinylpyrrolidone-vinyl acetate copolymer is 0.3 wt % to 2 wt %. Preferably, the amount of the polyvinylpyrrolidone-vinyl acetate copolymer is 0.5 wt % to 2 wt %.

In one of the embodiments, the aforementioned layered material may be used for adhering to the mucosa of eyes, ears, respiratory tract (e.g., nose, trachea), digestive tract (e.g., mouth, lips, tongue, gum, stomach, anus), and genital system (e.g., uterus), but is not limited thereto. In one of the embodiments, the mucosa may be endometrium, nasal mucosa, mucosa of digestive tract (e.g., oral mucosa, lingual mucosa, gastric mucosa), and eye mucosa, but is not limited thereto. In one of the embodiments, the aforementioned layered material is used to adhere to the oral mucosa. In one of the embodiments, the aforementioned layered material is used to adhere to the gum mucosa.

In one of the embodiments, the supportive layer of the patch is a water-resistant layer. In one of the embodiments, when the aforementioned layered material is coated on a water-resistant layer (e.g., a water-resistant film), its adhesive time to a glass slide under a mucosa-simulating condition (an aqueous environment with pH 7.4) may be prolonged. In one of the embodiments, the water-resistant layer may be a polyethylene terephthalate (PET) film, a thermoplastic polyurethane (TPU) film, a medical tape, etc.

In one of the embodiments, the supportive layer of the patch is a PET film, whose adhesive time to a glass slide under a mucosa-simulating condition (an aqueous environment with pH 7.4) is 12 minutes to 6 hours, 1 hour to 5 hours, or 2 hours to 4 hours. The PET film (the water-resistant film) may prolong the adhesive time.

In one of the embodiments, the supportive layer of the patch is a medical tape, whose adhesive time to a glass slide under a mucosa-simulating condition (an aqueous environment with pH 7.4) is 1 hour to 10 hours, 1 hour to 5 hours, or 2 hours to 4 hours.

In one of the embodiments, the supportive layer of the patch is a medical gauze, whose adhesive time to a glass slide under a mucosa-simulating condition (an aqueous environment with pH 7.4) is 1 minute to 1 hour, 10 minutes to 50 minutes, or 30 minutes to 40 minutes.

In one of the embodiments, the adhesive time is prolonged through increasing the thickness of the adhesive layer of the patch (i.e., the layered material of the present invention).

In one of the embodiments, the aforementioned adhesive layer of the patch is contacted with the mucosa. In one of the embodiments, the adhesive layer of the patch comprises the layered material for mucoadhesion of the present invention and an effective component.

In one of the embodiments, the aforementioned backing layer of the microneedle patch is composed of the layered material for macoadhesion of the present invention.

In one of the embodiments, the aforementioned adhesive layer of the microneedle patch comprises an effective component.

In one of the embodiments, the effective component comprised in the adhesive layer of the microneedle patch may be functional components such as drugs, growth factors, and nutrients. In one of the embodiments, the aforementioned effective component may be a functional component used for preventive health care, repair promotion, moisture maintenance, anti-inflammation, and soothing, but is not limited thereto. Specifically, the effective component used for preventive health care and repair promotion may be selected as stem cell extracts and their related substances, placenta extracts, growth factors, proteins, vitamin B, zinc, iron, potassium, vitamin A, vitamin C, vitamin D, and vitamin E, but is not limited thereto. The moisturizing component may be selected as ceramide, lecithin, glycerol, polysaccharides, hyaluronic acid, sodium hyaluronate, proteins, collagen, elastin, peptides, amino acids, citrate, uric acid, urea, glucose, sucrose, fructose, glycogen, glucosamine, mucopolysaccharides, lactates, phosphates, and eythyl-di-2-pyrrolidone-5-carboxylate, but is not limited thereto. The anti-inflammatory or soothing component may be selected as grape extracts, green tea extracts, ginkgo extracts, soy extracts, pomegranate extracts, ginger extracts, yeast extracts, coix extracts, R-alpha-lipoic acid, glucan, coenzyme Q10, superoxide (forward) dismutase (SOD), vitamin C and its derivatives, vitamin E and its derivatives, anti-inflammation agents, steroidal anti-inflammatory materials, non-steroidal anti-inflammatory materials, anti-pain and anti-inflammation materials and their analogs, vitamin B and its derivatives, licorice extracts, triterpenoids, madecassoside, asiaticoside, mucosa coating agents, glutathione, and lycopenemia, but is not limited thereto.

In one of the embodiments, the aforementioned microneedle patch may further comprise a water-resistant layer, and the water-resistant layer is disposed on the side of the backing layer which is free of connection with the base layer. In one of the embodiments, the aforementioned water-resistant layer may be a PET film, a TPU film, a medical tape, etc.

In one of the embodiments, the water-resistant layer of the microneedle patch is a PET film, whose adhesive time to a glass slide under a mucosa-simulating condition (an aqueous environment with pH 7.4) is 12 minutes to 6 hours, 1 hour to 5 hours, or 2 hours to 4 hours.

In one of the embodiments, the water-resistant layer of the microneedle patch is a medical tape, whose adhesive time to a glass slide under a mucosa-simulating condition (an aqueous environment with pH 7.4) is 1 hour to 10 hours, 1 hour to 5 hours, or 2 hours to 4 hours.

In one of the embodiments, the adhesive time is prolonged through increasing the thickness of the backing layer of the microneedle patch (i.e., the layered material of the present invention).

In one of the embodiments, the patch or the microneedle patch of the present invention may be applied to mucosal lesions, in which the mucosa may be the mucosa of eyes, ears, respiratory tract (e.g., nose, trachea), digestive tract (e.g., mouth, lips, tongue, gum, stomach, anus), and genital system (e.g., uterus), but is not limited thereto. In one of the embodiments, the mucosa may be endometrium, nasal mucosa, mucosa of digestive tract (e.g., oral mucosa, lingual mucosa, gastric mucosa), and eye mucosa, but is not limited thereto. In one of the embodiments, the mucosa may be the gum mucosa.

In one of the embodiments, the patch or the microneedle patch of the present invention may be applied to, but is not limited to, the following lesions located on the mucosa, periodontal disease, erythema multiforme, systemic lupus erythematosus, aphthous stomatitis, pemphigus, cold sores, oral cancer, angular cheilitis, and dental stomatitis, but is not limited thereto. In one of the embodiments, the aforementioned mucosa may have, but is not limited to, the following symptoms: erosion, ulceration, white lesion, petechiae (e.g., diffuse gastric mucosal petechiae), but is not limited thereto.

In one of the embodiments, the aforementioned microneedle patch is used for preventing or treating an oral disease. In one of the embodiments, the aforementioned oral disease is periodontal disease.

In one of the embodiments, the aforementioned microneedle patch is used for preventing or treating gingival recession. According to the present invention, gingival recession may be an early-onset of gingival recession or induced by factors such as heredity, irregular tooth alignment, periodontal disease, bruxism, denture compression, improper brushing behavior, smoking, and the like.

The layered material of the present invention in the oral environment is adhesive, so it may be used as an adhesive for the oral cavity, and may stably deliver the drugs of the patch or the microneedle patch into specific regions inside the oral cavity. The layered material is biodegradable and biocompatible and has good mechanical strength, and it may be combined and applied to different devices.

### BRIEF DESCRIPTION OF THE DRAWING(S)

FIG. 1 is a cross-sectional view of a microneedle patch of the present invention.

FIG. 2 is a schematic diagram of the preparation of the microneedle patch of the present invention.

FIG. 3 is a structural diagram of the master mold for preparing the microneedle patch of the present invention.

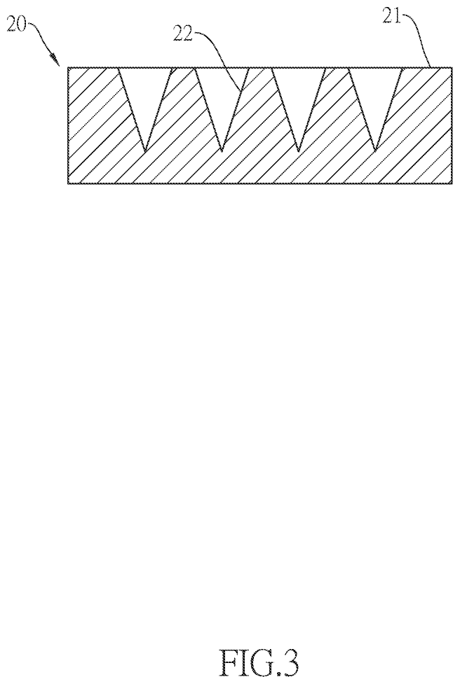

### DETAILED DESCRIPTION OF THE PREFERRED EMBODIMENTS

Followings are further explanations about the technical means of the present invention through the embodiments of the present invention.

### Illustrations of the Reagents

1. modified acrylic polymer: carbopol, purchased from Lubrizol Corporation, product name: Carbopol® Ultres 20 polymer, which is Acrylates/C10-30 Alkyl Acrylate Crosspolymer.

2. high-viscosity hydroxypropyl methylcellulose (1^{st} HPMC):
(1) 65SH-400, whose viscosity is 400 cP, purchased from Shin-Etsu Chemical Co., Ltd;
(2) 65SH-1500, whose viscosity is 1500 cP (@20° C., 2% solution), purchased from Shin-Etsu Chemical Co., Ltd; 8
(3) 90SH-4000, whose viscosity is 4000 cP (@20° C., 2% solution), purchased from Shin-Etsu Chemical Co., Ltd;
(4) 90SH-4000SR, whose viscosity is 4000 cP (@20° C., 2% solution), purchased from Shin-Etsu Chemical Co., Ltd;
(5) 65SH-4000, whose viscosity is 4000 cP, (@20° C., 2% solution) purchased from Shin-Etsu Chemical Co., Ltd;
(6) 60SH-4000, whose viscosity is 4000 cP (@20° C., 2% solution), purchased from Shin-Etsu Chemical Co., Ltd;
(7) 60SH-10000, whose viscosity is 10000 cP (@20° C., 2% solution), purchased from Shin-Etsu Chemical Co., Ltd. 18

3. low-viscosity hydroxypropyl methylcellulose (2^{nd} HPMC):
(1) PHARMACOAT 603, whose viscosity is 3 cP (@20° C., 2% solution), purchased from Shin-Etsu Chemical Co., Ltd;
(2) SB-4, whose viscosity is 4 cP (@20° C., 2% solution), purchased from Shin-Etsu Chemical Co., Ltd;
(3) PHARMACOAT 645, whose viscosity is 4.5 cP (@20° C., 2% solution), purchased from Shin-Etsu Chemical Co., Ltd;
(4) PHARMACOAT 606, whose viscosity is 6 cP (@20° C., 2% solution), purchased from Shin-Etsu Chemical Co., Ltd;
(5) PHARMACOAT 615, whose viscosity is 15 cP (@20° C., 2% solution), purchased from Shin-Etsu Chemical Co., Ltd;
(6) METOLOSE 65SH-50, whose viscosity is 50 cP (@20° C., 2% solution), purchased from Shin-Etsu Chemical Co., Ltd;
(7) METOLOSE 60SH-50, whose viscosity is 50 cP (@20° C., 2% solution), purchased from Shin-Etsu Chemical Co., Ltd.

4. polyvinylpyrrolidone-vinyl acetate copolymer, (PVP/VA), purchased from BASF Corporation, product name: Kollidon.

### Preparation Example 1: Preparing the Layered Material of the Present Invention

According to the compositions shown in Table 1, the aforementioned modified acrylic polymer was mixed with a variety of hydroxypropyl methylcellulose having different viscosities to prepare the layered material composition liquid for each Example.

TABLE 1
The formulations of the layered material composition
liquid from Examples A1 to A5 and B1 to B5

| | | | hydroxypropyl | | |
|---|---|---|---|---|---|
| | modified acrylic polymer | | methylcellulose | | solid |
| | | weight | | weight | content |
| | Type | ratio | type | ratio | (% w/v) |
| Example | modified acrylic | 1 | 2^{nd} HPMC | 2 | 3 |
| A1 | polymer | | | | |
| Example | modified acrylic | 1 | 2^{nd} HPMC | 1 | 3 |
| A2 | polymer | | | | |
| Example | modified acrylic | 2 | 2^{nd} HPMC | 1 | 3 |
| A3 | polymer | | | | |
| Example | modified acrylic | 2 | 2^{nd} HPMC | 1 | 2 |
| A4 | polymer | | | | |
| Example | modified acrylic | 2 | 2^{nd} HPMC | 1 | 1.5 |
| A5 | polymer | | | | |
| Example | modified acrylic | 1 | 1^{st} HPMC | 2 | 3 |
| B1 | polymer | | | | |
| Example | modified acrylic | 1 | 1^{st} HPMC | 2 | 2 |
| B2 | polymer | | | | |
| Example | modified acrylic | 1 | 1^{st} HPMC | 2 | 1.5 |
| B3 | polymer | | | | |
| Example | modified acrylic | 1 | 1^{st} HPMC | 1 | 3 |
| B4 | polymer | | | | |
| Example | modified acrylic | 2 | 1^{st} HPMC | 1 | 3 |
| B5 | polymer | | | | |

The low-viscosity hydroxypropyl methylcellulose (2^{nd} HPMC) of Examples A1 to A5 and the high-viscosity hydroxypropyl methylcellulose (1^{st} HPMC) of Examples B1 to B5 may be randomly selected from the aforementioned reagents.

### Test Example 1: Testing the Adhesive Properties of the Layered Material of the Present Invention

The layered material composition liquid in the aforementioned Examples was spray-coated on a PET film (1139 OHP Grade A overhead projection film, from Master Business Systems) as a water-resistant layer. The PET film had a thickness of about 0.1 mm, and the spraying thickness of the layered material was 25 mm. After drying in an oven at 30° C. for 6 hours, a PET film coated with the layered material was produced, whose layer thickness after drying the layered material was 0.01 mm. If other thickness of the layered material is required, one should adjust the spraying thickness and the drying time. After drying, the aforementioned PET film coated with the layered material was cut into samples of 2 centimeters (cm)×2 cm for an adhesive test.

Phosphate buffered saline (PBS) of 0.1 milliliters (mL) was dripped onto one side of a glass slide surface. The layered material of the aforementioned sample was faced downward to cover the PBS and part of the glass slide surface, making adhesion between the sample and the glass slide occur through the layered material of the present invention under an aqueous environment at pH 7.4. A heavy object of 700 grams was pressed on the other side of the PET film of the aforementioned sample for 30 seconds to tighten the adhesion. After that, the end of the glass slide adhered to the sample was placed into a vessel filled with PBS of pH 7.4, making the sample and the part of the glass slide to which the sample adhered totally immersed into the PBS buffer, and the glass slide (and the samples on the glass slide) was placed vertically to the bottom of the vessel. The adhesive time was recorded from the immersion of the sample to the sample falling off from the glass slide. The aforementioned PBS buffer is used to simulate an environment of the oral cavity with saliva (pH ranging from 6.2 to 7.6). The adhesive time of the sample made from the aforementioned Examples of the layered material is shown in Table 2.

TABLE 2
the thickness of the layered material of the Examples of the
present invention and their adhesive time to the glass slide

| | the thickness of the | the adhesive | |
|---|---|---|---|
| | layered material | time | |
| Example A1 | 0.01 mm | 1.3 | hours |
| Example A2 | 0.01 mm | 1.5 | hours |
| Example A3 | 0.01 mm | >2 | hours |
| Example A4 | 0.01 mm | >2 | hours |
| Example A5 | 0.01 mm | >2 | hours |
| Example B1 | 0.01 mm | >2 | hours |
| Example B2 | 0.01 mm | >2 | hours |
| Example B3 | 0.01 mm | >2 | hours |
| Example B4 | 0.03 mm | >2 | hours |
| Example B5 | 0.04 mm | >2 | hours |

Furthermore, by using samples of Examples B2 and B3 to conduct the adhesive test as aforementioned, but replacing the PET film with a medical gauze (non-water-resistant layer) or an adhesive side of a medical tape (a 3M™ medical single-sided tape 1526 comprising a polypropylene film of about 0.09 mm and an adhesive layer of about 0.04 mm, which served as a water-resistant layer), the aforementioned water-resistant and non-water-resistant material were respectively combined with the layered material of Examples of the present invention, and the combined materials were tested to see their influences to the adhesive time to the glass slide. The test result is shown in Table 3. In this test experiment, the layered material had a thickness of 0.01 mm.

TABLE 3
the effect of the types of the combined materials with
the layered material of Examples of the present invention
to the adhesive time to the glass slide

| the material | | Medical tape |
|---|---|---|
| being | medical | (combined on the |
| combined | gauze | adhesive side) |
| Example B2 | 40 minutes | >2 hours |
| Example B3 | N/A | >2 hours |

As shown in Tables 2 and 3, when the layered material of Examples of the present invention was combined with a water-resistant layer (PET film, medical tape, etc.), the adhesion to the glass slide under a simulated oral environment (an aqueous environment with pH 7.4) lasted for more than 1.3 hours, ensuring the fixation under a mucosa-simulating condition. The medical gauze was not a water-resistant layer, whose effect was simply the same as that of the layered material of Examples of the present invention.

### Preparation Example 2: Preparing a Microneedle Patch

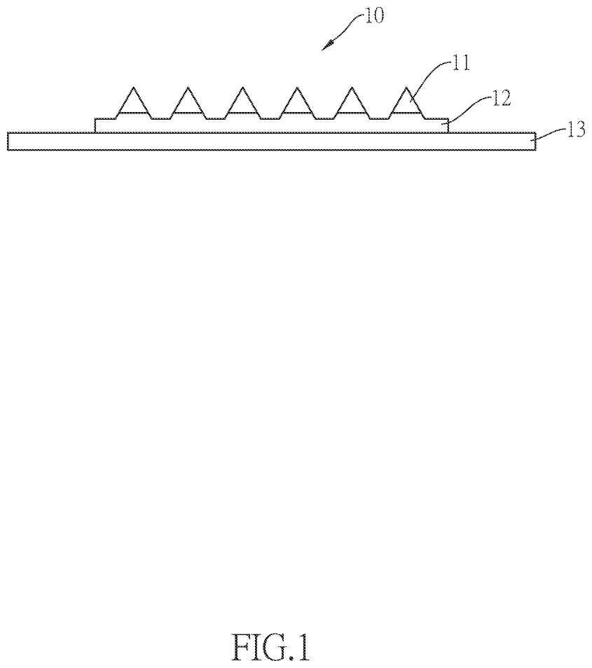

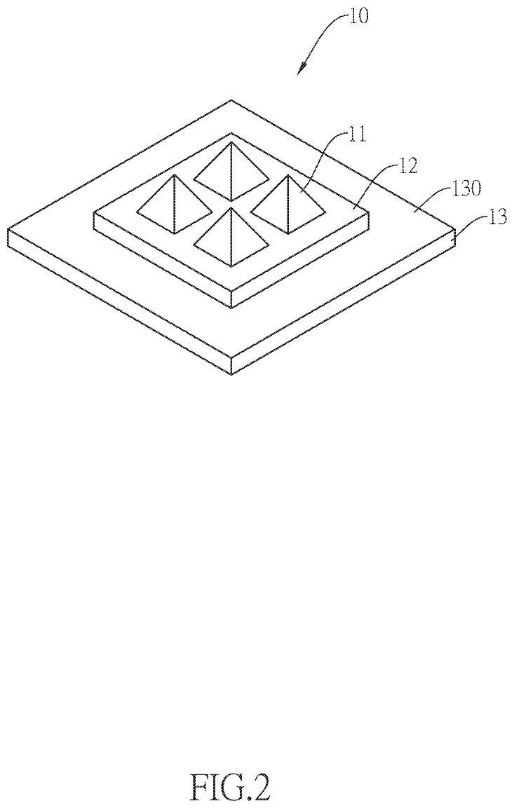

FIGS. 1 and 2 show the microneedle patch 10 of the present invention, comprising a needle layer 11, a base layer 12, and a backing layer 13, wherein the backing layer 13 comprises the layered material for mucoadhesion of the present invention, the base layer 12 is disposed between the needle layer 11 and the backing layer 13, and the backing layer 13 comprises at least one protrusion part 130 which is free of connection with the base layer 12. In the preparation example, the backing layer 13 was comprised of the layered material for mucoadhesion of the present invention.

During production, the microneedle patch 10 comprising the layered material for mucoadhesion of the present invention could be produced through the following procedures.

A master mold 20 was provided. As shown in FIG. 2, the master mold 20 comprises a datum plane 21 and multiple holes 22, the multiple holes 22 are downwardly caved from the datum plane 21, and the multiple holes 22 are concavely arranged in an array on the master mold 20. In this example, the material of the master mold 20 was polydimethylsiloxane (PMDS). The hole density of the master mold 20 was 289 holes per square centimeter (cm²); the size of the array of holes was 1.5 cm×1.5 cm; and each hole 22 was square cone-shaped, with a depth (i.e., the vertical distance between the tip of the hole 22 and the datum plane 21) of 300 micrometers (μm) and the maximum width (i.e., the maximum internal diameter of the horizontal surface of the hole 22, which is flush with datum plane 21) was 150 μm.

Afterward, by slot-die coating method, the needle composition liquid was squeezed out from a slit nozzle of a slot-die coating head with a coating gap of 1000 μm and a coating rate of 3 meters per minute (m/min). The squeezed-out needle composition liquid was coated inside the multiple holes 22 of the master mold 20, making the needle composition liquid completely filled in the multiple holes 22 of the master mold 20. Until the liquid level was as high as the datum plane 21, a cavity 20 filled with the needle composition liquid was obtained. The needle composition liquid was a 20 wt % solution comprising the copper peptide and methyl vinyl ether-maleic anhydride copolymer, i.e., the needle composition liquid contained 80 wt % of water and 20 wt % of a mixture of a copper peptide and methyl vinyl ether-maleic anhydride copolymer. The needle composition liquid had a viscosity of 40 cP under 25° C., 1 S⁻¹ and had a surface tension of 30 dyne/cm.

Then the master mold 20 filled with the needle composition liquid was continuously dried at 30° C. for 1 hour, which dried the needle composition liquid and formed a needle. Therefore, a master mold 20 with a needle layer 11 was obtained, wherein the needle layer 11 was formed by the needle. The needle composition liquid would dent inwardly after drying, and the formed bottom surface of the needle was lower than the datum plane 21.

The formulations for preparing a base composition liquid are shown in Table 4, wherein the unit of each component of the base composition liquids 1 and 2 is weight percentage (wt %).

TABLE 4
the compositions and properties of the base composition liquid

| | Composition | | | | |
|---|---|---|---|---|---|
| | 1^{st} | 2^{nd} | | solid | |
| | HPMC | HPMC | PVP/VA | content | viscosity |
| base | 1 wt % | 0.25 wt % | 2 wt % | 3.25 wt % | 235.8 cP |
| composition | | | | | |
| liquid 1 | | | | | |
| base | 1 wt % | 0.5 wt % | 0.25 wt % | 1.75 wt % | 199.2 cP |
| composition | | | | | |
| liquid 2 | | | | | |

The aforementioned base composition liquid 1 or 2 was coated inside the multiple holes 22 of the master mold 20 by the slot-die coating method with a coating gap of 1600 μm and a coating rate of 3 m/min. The PDMS master mold 20 with the base composition liquid was placed inside a vacuum oven with a pressure of 35 torr for air extraction. The base composition liquid 1 or 2 then entered into the holes 22 of the master mold 20 and formed a base layer 12 on the needle layer 11 of the cavity 20. The base layer 12 was continuously dried at 30° C. under 45% to 75% relative humidity for 1 hour, which made the aforementioned base composition liquid dry and form a base layer 12 with a water content of less than 20%.

Any of the layered material composition liquid of the present inventions as shown in Table 1 formed a backing layer 13 on the base layer 12 by the slot-die coating with a coating gap of 1600 μm and a coating rate of 3 m/min. The backing layer 13 was continuously dried at 30° C. under 45% to 75% relative humidity for 1 hour, which made the aforementioned layered material composition liquid dry and form a backing layer 13 with a water content of less than 20%. The aforementioned backing layer 13 had a thickness of 0.1 mm to 1 mm. At last, a microneedle structure having the needle layer 11, the base layer 12 and the backing layer 13 were demolded from the PDMS master mold, and thus the production of the microneedle patch 10 was completed.

The above description is merely some specific embodiments of the present invention. The various examples shall not limit the essential content of the present invention. A person having ordinary skill in the art, after reading the specification, can make modifications or variations of the specific embodiments as abovementioned without departing from the essence and scope of the present invention.

1. A layered material for mucoadhesion, comprising a modified acrylic polymer and hydroxypropyl methylcellulose, wherein a weight ratio of the modified acrylic polymer: the hydroxtpropyl methylcellulose is 1:4 to 4:1, and the layered material has a thickness of 0.0001 millimeters to 100 millimeters.

2. The layered material as claimed in claim 1, wherein the layered material is obtained by drying a layered material composition liquid comprising the modified acrylic polymer and the hydroxypropyl methylcellulose, wherein the layered material composition liquid has a solid content of 0.5 weight percent to 30 weight percent.

3. The layered material as claimed in claim 1, wherein the modified acrylic polymer is a hydrophobically-modified cross-linked acrylate copolymer.

4. The layered material as claimed in claim 1, wherein the hydroxypropyl methylcellulose has a viscosity of 1 cP to 10000 cP.

5. The layered material as claimed in claim 4, wherein the hydroxypropyl methylcellulose has a viscosity of 400 cP to 10000 cP, and the weight ratio of the modified acrylic polymer: the hydroxypropyl methylcellulose is 1:1.5 to 1:4.

6. The layered material as claimed in claim 4, wherein the hydroxypropyl methylcellulose has a viscosity of 400 cP to 10000 cP, and the weight ratio of the modified acrylic polymer: the hydroxypropyl methylcellulose is 2:1 to 3.5:1.

7. The layered material as claimed in claim 4, wherein the hydroxypropyl methylcellulose has a viscosity of 1 cP to 100 cP, and the weight ratio of the modified acrylic polymer: the hydroxypropyl methylcellulose is 1:1.5 to 4:1.

8. A patch, comprising a supportive layer and an adhesive layer, wherein the adhesive layer comprises the layered material of claim 1, and the adhesive layer is disposed on one side of the supportive layer.

9. A patch, comprising a supportive layer and an adhesive layer, wherein the adhesive layer comprises the layered material of claim 5, and the adhesive layer is disposed on one side of the supportive layer.

10. A patch, comprising a supportive layer and an adhesive layer, wherein the adhesive layer comprises the layered material of claim 6, and the adhesive layer is disposed on one side of the supportive layer.

11. A patch, comprising a supportive layer and an adhesive layer, wherein the adhesive layer comprises the layered material of claim 7, and the adhesive layer is disposed on one side of the supportive layer.

12. A microneedle patch, comprising a needle layer, a base layer, and a backing layer, wherein the backing layer comprises the layered material of claim 1; the base layer is disposed between the needle layer and the backing layer; and the backing layer comprises at least one protrusion part which is free of connection with the base layer.

13. The microneedle patch as claimed in claim 12, wherein the needle layer comprises an effective component.

14. The microneedle patch as claimed in claim 12, wherein the microneedle patch further comprises a water-resistant layer, and the water-resistant layer is disposed on the side of the backing layer which is free of connection with the base layer.

15. A microneedle patch, comprising a needle layer, a base layer, and a backing layer, wherein the backing layer comprises the layered material of claim 2; the base layer is disposed between the needle layer and the backing layer; and the backing layer comprises at least one protrusion part which is free of connection with the base layer.

16. A microneedle patch, comprising a needle layer, a base layer, and a backing layer, wherein the backing layer comprises the layered material of claim 3; the base layer is disposed between the needle layer and the backing layer; and the backing layer comprises at least one protrusion part which is free of connection with the base layer.

17. A microneedle patch, comprising a needle layer, a base layer, and a backing layer, wherein the backing layer comprises the layered material of claim 4; the base layer is disposed between the needle layer and the backing layer; and the backing layer comprises at least one protrusion part which is free of connection with the base layer.

18. A microneedle patch, comprising a needle layer, a base layer, and a backing layer, wherein the backing layer comprises the layered material of claim 5; the base layer is disposed between the needle layer and the backing layer; and the backing layer comprises at least one protrusion part which is free of connection with the base layer.

19. A microneedle patch, comprising a needle layer, a base layer, and a backing layer, wherein the backing layer comprises the layered material of claim 6; the base layer is disposed between the needle layer and the backing layer; and the backing layer comprises at least one protrusion part which is free of connection with the base layer.

20. A microneedle patch, comprising a needle layer, a base layer, and a backing layer, wherein the backing layer comprises the layered material of claim 7; the base layer is disposed between the needle layer and the backing layer; and the backing layer comprises at least one protrusion part which is free of connection with the base layer.
